(19) United States
(12) Patent Application Publication
(10) Pub. No.: US 2004/0210256 A1
(43) Pub. Date: Oct. 21, 2004

(54) Methods and apparatus for decreasing incidences of early recurrence of atrial fibrillation

(75) Inventors: Shaileshkumar V. Musley, Blaine, MN (US); Eduardo N. Warman, Maple Grove, MN (US); David E. Euler, Minnetonka, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/417,978

(22) Filed: Apr. 17, 2003

(51) Int. Cl. A61N 1/39

(52) U.S. Cl. 607/5

(57) ABSTRACT

Implantable cardioverter/defibrillators (ICDs) providing atrial cardioversion/defibrillation (C/D) shock therapies in response to detected atrial tachyarrhythmias, particularly atrial fibrillation (AF) or in response to a patient command generated by a patient experiencing AF symptoms are disclosed. The early recurrence of atrial fibrillation (ERAF) following delivery of an atrial C/D shock is detected and delivery of any subsequent atrial C/D shock therapies is delayed for an ERAF sustained duration.

### FIELD OF THE INVENTION

This invention relates to implantable cardioverter/defibrillators (ICDs) providing atrial cardioversion/defibrillation (C/D) shock therapies in response to detected atrial tachyarrhythmias or patient initiated commands and particularly to methods and apparatus for recognizing incidences of early recurrence of atrial fibrillation (ERAF) following delivery of a C/D shock and delaying delivery of subsequent atrial C/D shock therapies to a more efficacious time.

### BACKGROUND OF THE INVENTION

By way of definition, tachyarrhythmias are episodes of high rate cardiac depolarizations, typically occurring in one chamber of the heart but which may be propagated from one chamber to the other, and are distinguished from sinus tachycardias that physiologically accompany exercise to provide adequate cardiac output. Tachyarrhythmias that are excessively high in rate and chaotic can compromise cardiac output from the affected chamber(s), leading to loss of consciousness and death, in the case of ventricular fibrillation, or weakness and dizziness, in the case of atrial fibrillation or flutter and non-sinus atrial and ventricular tachycardias. High rate atrial and ventricular tachycardias may exhibit a more organized rhythm but also may disable the patient and can progress to ventricular fibrillation if untreated.

In the field of automatic implantable arrhythmia control devices, the term “cardioversion” or “cardioverter” refers to the process of and device for discharging relatively high-energy electrical shocks in excess of 1.0 Joule into or across cardiac tissue to arrest or “cardiovert” a tachyarrhythmia of a cardiac chamber. Delivery of cardioversion shocks may or may not be synchronized with a cardiac depolarization or rhythm and may be applied to arrest a malignant atrial or ventricular tachycardia or fibrillation with a programmable shock energy. The termination of high rate tachycardias with lesser energy electrical shocks or burst pacing has also been referred to as “cardioversion”. The arrest of atrial or ventricular fibrillation by higher energy shocks is referred to as “defibrillation”, although defibrillation has been characterized in the past as a form of cardioversion. The first implanted implantable arrhythmia control device for providing synchronized cardioversion shocks or and unsynchronized defibrillation shocks was designated as an “automatic implantable cardioverter/defibrillator” (AICD). The first implanted implantable arrhythmia control device for providing staged therapies of anti-tachyarrhythmia pacing, synchronized cardioversion shocks and unsynchronized defibrillation shocks was designated as a “pacemaker/cardioverter/defibrillator” (PCD). Most recently, more complex implantable arrhythmia control devices have been developed adding dual chamber and/or right and left heart chamber pacing and sensing capabilities. All such single, dual and left and right heart-chamber implantable arrhythmia control devices are currently referred to ICDs. In the following description and claims, it is to be assumed that the terms “cardioversion” and “defibrillation” and variants thereof are interchangeable, and that use of one term is inclusive of the other device or operation, unless specific distinctions are drawn between them in the context of the use. For convenience, the term “cardioversion” or “C/D” will be used herein to encompass cardioversion and defibrillation unless a form of defibrillation therapy is specifically referred to.

Generally a relatively high-energy C/D shock must be applied to counter high rate malignant ventricular tachycardia or ventricular fibrillation (herein referred to collectively as VF) and save the patient's life. The high-energy ventricular C/D shock is usually not perceived by the patient because of the loss of consciousness shortly following onset of the arrhythmia. Accuracy of diagnosis and delivery of a C/D shock having sufficient energy to cardiovert VF as quickly as possible are paramount concerns because the efficacy of the ventricular C/D shock decreases with time lapse from onset of the symptoms.

Atrial tachyarrhythmia episodes, e.g., atrial fibrillation or atrial flutter (collectively “AF”), decrease cardiac output but are usually not life threatening; however, the repeated AF episodes weaken and discourage the patient. Drug treatments having limited success are usually prescribed, and the patients are frequently hospitalized and undergo frequent atrial C/D shock therapy applied externally to at least temporarily alleviate the symptoms. There is concern that the delivered atrial C/D shock might itself induce VF leading to the death of the patient. In the hospital setting, the patient is carefully monitored, and induced VF may be defibrillated. However, the clinical procedure still entails enough risk that drug therapies are preferred, and atrial C/D shocks are applied only after other therapies fail.

Patients experiencing high rate atrial tachyarrhythmias including atrial tachycardia (AT) and AF episodes typically do not lose consciousness, and the symptoms of an atrial tachyarrhythmia episode are readily recognizable by the patient. Therefore, patient-activated atrial ICDs were first developed and clinically implanted in such patients as set forth in U.S. Pat. Nos. 3,952,750, 5,439,481, 5,931,857, and 6,068,651, for example. Patient activated atrial ICDs comprise an ICD implantable pulse generator (IPG) implanted subcutaneously, and a set of C/D leads extending from the atrial ICD IPG to at least one C/D electrode disposed in operative relation to the atria. The patient is provided with an external, manually actuated, patient activator that the patient experiencing atrial tachyarrhythmia symptoms can employ to downlink telemetry transmit a command that is received by the ICD IPG to initiate delivery of an atrial C/D shock. The patient initiated atrial C/D shock is then delivered by the ICD through the atrial C/D electrodes disposed about the atria.

Complications of blood clotting in the atria can arise from prolonged AF episodes, and the belated delivery of an AF C/D shock therapy can cause such clots to dislodge from the atria and travel into the brain or lungs or other locations in the body endangering the patient's life. It is thus generally agreed that delivery of atrial C/D shocks should not be attempted without the use of anticoagulants if the patient has been in AF fibrillation for more than 48 hours, and it is recommended that anticoagulants be used if the patient has been in AF fibrillation for more than 24 hours. The above-referenced 651 patent discloses an atrial ICD that functions in a semi-automatic mode by detecting the onset or AF, alerting the patient, and timing out a patient delay until the patient initiates atrial C/D shock delivery using the external patient activator, and locks out such delivery if the patient delay exceeds a safe delivery window that can be selected between 6 and 72 hours.

The concern of possible inducement of fatal VF has slowed the development and acceptance of atrial ICDs that automatically detect and deliver C/D shocks to the atria. Nevertheless, a great deal of research and development has also been undertaken over the years to develop atrial ICDs that can automatically safely discriminate atrial and ventricular tachyarrhythmias, particularly AF and VF, and deliver appropriate and safe atrial C/D shock therapies in response to a detected AF episode. One possible approach that has been implemented combines AF and VF detection and C/D shock delivery capabilities in a single, dual chamber ICD so that discrimination between AF and VF is enhanced, and so that any VF episode induced by an applied atrial C/D shock can be automatically detected and cardioverted by an applied ventricular C/D shock.

In clinically implanted and in proposed atrial ICDs, atrial C/D electrodes are implanted in operative relation to the atria, typically through a transvenous approach disposing one elongated coil, C/D electrode in the region of the superior vena cava (SVC) and another elongated coil, C/D electrode in the right ventricle (RV) or in the coronary sinus (CS). A further subcutaneous C/D electrode typically comprising an exposed surface of the hermetically sealed housing or “can” of the ICD IPG is also typically employed. The atrial ICD detects AT and AF episodes and delivers programmed AT and AF shock therapies through programmed atrial C/D pathways including between the SVC, RV and CAN C/D electrodes wherein selected ones of the SVC, RV (or CS) and CAN C/D electrodes are employed alone or tied together as anode and cathode electrodes. For example, an ICD coupled with RV, SVC and CAN C/D electrodes may be programmed to deliver the C/D shock therapy in a pathway employing the RV C/D electrode as the anode electrode and the SVC+CAN C/D electrodes as the cathode electrode.

A certain atrial C/D shock energy (the atrial cardioversion threshold) is necessary to successfully cardiovert atria that are in AF. Typically reported atrial cardioversion thresholds (in humans) of 4-12 Joules are required in an atrial C/D pathway between SVC+CAN and RV C/D electrodes. Other atrial C/D electrode pathways may require up to 30 Joules (in humans) to reliably cardiovert the atria. Conscious patients subjected to atrial C/D shocks in these ranges often experience significant discomfort and intolerable pain. Patients resort to pain killers and in certain cases postpone activating the patient activated atrial C/D shock therapy, preferring to tolerate the AF symptoms instead.

For these reasons, it is generally believed desirable to reduce the cardioversion energy threshold as much as possible to reduce the pain associated with atrial C/D shock therapy to an acceptable level. A desirable atrial cardioversion energy threshold is generally stated to be 2 joules or less, preferably 1 joule or less. Studies have focused on reducing the atrial cardioversion energy threshold by altering the shape (waveform) of the delivered shock pulse or the locations of the C/D electrodes (electrode configuration) through which atrial C/D shocks are applied. Many other proposals have been made to alleviate the surprise and discomfort experienced by the patient.

In one approach, implemented in or suggested for inclusion in both atrial and ventricular ICDs set forth in U.S. Pat. Nos. 5,332,400 and 5,630,838, an audible or subcutaneous electrical stimulation or other warning that can be perceived by the patient is generated by the atrial or ventricular ICD in advance of delivery of the atrial C/D shock. The patient can mentally brace for delivery and perhaps move to a resting place or safe position before the atrial or ventricular C/D shock is delivered. Other approaches that are suggested in U.S. Pat. Nos. 5,893,881 and 6,438,418, for example, for reducing the perceived pain attendant to delivery of atrial C/D shocks include the delivery of pain reducing drugs or electrical nerve stimulation prior to atrial C/D shock delivery.

In commonly assigned U.S. Pat. No. 5,630,834, a dual chamber atrial ICD is disclosed that has the ability to determine whether the patient is likely to be asleep. Atrial C/D shocks having shock energy levels that would normally be painful to the patient are delivered only in response to detected AF episodes at times that the patient is likely to be asleep. At other times, lower energy atrial C/D shocks can be delivered in response to detected AF episodes. The fundamental hypothesis is that lowering of the cardioversion energy threshold will permit atrial cardioversion with weaker shocks and thereby decrease the pain associated with these shocks in patients. This method may require a patient to remain in AF for many hours until the patient falls asleep. Thus it is not practical for some patients who become symptomatic shortly after the onset of AF or for patients with ventricular arrhythmias who typically require treatment as soon as possible after the onset of the arrhythmia. Further, some patients have reported being awakened from sleep by painful and startling ICD shocks. Thus, administration of shocks during sleep is painful in some patients. In addition, a patient's knowledge that he/she may be shocked while asleep may result in anticipatory anxiety that interferes with sleep. Despite these drawbacks, the option is provided to the physician to program a “daily availability period” for delivery of atrial C/D shock therapy in the MEDTRONIC® GEM III AT® Model 7276 IPG for those patients who can benefit from limiting delivery to anticipated sleep time.

Most patients receiving such atrial C/D shock therapy continue to suffer because these attempts to alleviate the pain and discomfort of atrial C/D shocks have not yet proven to be sufficiently efficacious. As long as pain and discomfort accompany each such shock delivery, it is preferable to minimize the number and frequency of delivered painful atrial C/D shocks that are delivered. The delivered atrial C/D shock can either be successful or unsuccessful in cardioverting the AF episode as determined by success criterion defined in the operating system of the ICD. It is preferable to minimize the number of delivered unsuccessful atrial C/D shocks.

For example, typical ICD IPGs, e.g., the above-referenced MEDTRONIC® GEM III AT® IPG, can be programmed to deliver a “back-up” second or subsequent atrial C/D shock(s) typically having progressively increasing shock energy than the preceding delivered atrial C/D shock(s) if the earlier delivered atrial C/D shock proves to be unsuccessful. Studies have shown that a subsequent atrial C/D shock, delivered upon determination that the earlier delivered atrial C/D shock was not successful, is more painful to the patient than the earlier delivered atrial C/D shock. Moreover, the likelihood that a second or subsequent delivered atrial C/D shock will enjoy success even if it is increased in shock energy is not good, particularly if the atrial C/D shock energy is programmed to minimize the pain perceived by the patient. Unfortunately, the patient can be subjected to two or more painful atrial C/D shocks without enjoying the successful termination of the AF episode.

Therefore, many clinicians do not program the ICD IPG to provide any back-up atrial ICD therapies in response to a determination that the first delivered atrial C/D shock was not successful. Moreover, many clinicians program the ICD IPG to require the time-out of a minimum “AF therapy delay” since the preceding delivered, successful or unsuccessful, atrial C/D shock before an automatic or patient initiated delivery of a subsequent atrial C/D shock is allowed. To accomplish this, the maximum number of atrial C/D shocks that can be delivered in a 24 hour period can be programmed in use of the above-referenced MEDTRONIC® GEM III AT IPG. Presently, it is recommended in use of the above-referenced MEDTRONIC® GEM III AT IPG that only a single atrial C/D shock be delivered during the programmed daily availability period in response to the automatic detection of an AF episode. The delivery of an atrial C/D at least once every 24 hours in response to detection of an AF episode is provided to avoid the possibility that a sustained AF episode is in progress and could result in blood clotting as noted above.

One of the reasons for recommending delivery of only a single atrial C/D shock in response to a detected AF episode during a 24 hour period arises from the recognition that the determination that the AF episode has been successfully cardioverted following an algorithm of the ICD operating system can be followed rather quickly by the recurrence of an AF episode. See, for example, C. Timmermans et al., “Immediate Reinitiation of Atrial Fibrillation Following Internal Atrial Defibrillation” J. Cardiovasc. ElectroPhysiol. 1998;9:122-9; H-F Tse et. al., “Incidence and modes of onset of early reinitiation of atrial fibrillation after successful internal cardioversion, and its prevention by intravenous sotalol” HEART 1999;82:319-24; J. Sra et. al., “Spontaneous Reintiation of Atrial Fibrillation Following Transvenous Atrial Defibrillation” PACE 1998;21:1105-10; H-F Tse et. al., “Atrial pacing for suppression of early reinitiation of atrial fibrillation after successful internal cardioversion” Eur. Heart J. 2000;21:1167-76; R. G. Teleman et. al., “Early Recurrences of Atrial Fibrillation After Electrical Cardioversion: A Result of Fibrillation Induced Electrical Remodeling of the Atria?” J. Am. Coll. Cardiol. 1998;31:167-73; and D. Schwartzman et. al., “Early Recurrence of Atrial Fibrillation Following Ambulatory Shock Conversion” J. Am. Coll. Cardiol. 2002;40:93-9.

Such an early recurrence of atrial fibrillation or ERAF can develop after the ICD algorithm has declared the delivered atrial C/D shock to be successful or the ERAF can be mistaken by the ICD algorithm as evidence that the delivered atrial C/D shock was unsuccessful. In either case, an ICD that is programmed to allow deliver of a second or a further first atrial C/D shock may deliver an ineffectual atrial C/D shock that can be again mistakenly determined to be successful or unsuccessful due to a further ERAF, and the process could continue until the maximum number of programmed atrial C/D shocks are delivered. Therefore, as noted above, it is recommended that clinicians allow only a single atrial C/D shock be delivered in a programmed daily availability period in response to the automatic detection of an AF episode. However, this imposed limitation denies the possibility that a second delivered atrial C/D shock delivered within the programmed daily availability period may prove effective to cardiovert either an unsuccessfully cardioverted ongoing AF episode or an ERAF that persists over time.

As noted in the above-referenced Schwartzman article, ERAF tends to commence most frequently within minutes of the delivery of an atrial C/D shock. Moreover, incidences of ERAF are strongly related to AF episodes that are relatively short in duration, i.e., less than 3 hours in duration. Thus, the delivery of a second atrial C/D shock into an ERAF shortly after delivery of an earlier patient initiated or automatically initiated atrial C/D shock may prove ineffective and can actually cause the ERAF to recur.

Therefore, a need exists for providing an atrial ICD IPG that can recognize and appropriately respond to ERAF episodes and that can be operated either automatically or manually to maximize the delivery of effective atrial C/D shock therapies and reduce incidences of ERAF that may be provoked by such automatic or manual delivery of atrial C/D shock therapies in order to enhance efficacy and reduce patient pain and discomfort.

### SUMMARY OF THE INVENTION

In accordance with the present invention, the AF ICD is programmed or otherwise configured to provide more than one atrial C/D shock in any AF therapy delay period, and ERAF is automatically detected following the automatic or manually initiated delivery of an atrial C/D shock. The delivery of any further atrial C/D shock is delayed for an ERAF sustained duration that tends to minimize the possibility that ERAF will follow the subsequent atrial C/D shock. The ERAF sustained duration is preferably programmed to provide a delay that operates optimally for the patient to avoid ERAF.

In an automatic operating mode, the subsequent atrial C/D shock can only be delivered after time-out of the ERAF sustained duration at the programmed atrial C/D shock energy and if AF is still present as determined automatically.

In the manual operating mode, any further patient commands to deliver atrial C/D shock therapy during time out of the ERAF sustained duration are ignored. The patient can manually attempt to initiate delivery of the subsequent atrial C/D shock at the atrial C/D shock energy that the patient is allowed to deliver, which may be a lower programmed shock energy than the programmed shock energy of the automatically delivered subsequent atrial C/D shock. Preferably, the patient is alerted when ERAF is declared following delivery of the initial atrial C/D shock, and the patient learns through experience that the subsequent atrial C/D shock will not be delivered immediately. The subsequent atrial C/D shock is only delivered after time-out of the ERAF sustained duration if the patient again initiates delivery of the atrial C/D shock using the patient activator.

An ICD that is otherwise operating in an automatic AF detection and atrial C/D shock therapy delivery mode as described above can also be operated by the patient using the patient activator, if the patient is provided with a patient activator.

Advantageously, these responses to ERAF avoid delivering a subsequent atrial C/D shock that will likely be unsuccessful. Unnecessary patient pain is eliminated and the likelihood of inducing a further ERAF is reduced.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic illustration of an atrial and ventricular ICD IPG adapted to be implanted in a patient's chest with endocardial leads transvenously introduced into the right heart chambers whereby atrial pacing pulses are delivered during an unstable VT episode precedent to the delivery of an anti-tachycardia therapy to enhance hemodynamic function; in accordance with the invention;

FIG. 2 is a block diagram of an exemplary ICD IPG operating system in which the present invention may be practiced;

FIG. 3 is a simplified flow chart illustrating the method of the present invention practiced in an atrial ICD operating in an automatic mode;

FIG. 4 is a simplified flow chart illustrating the method of the present invention practiced in an atrial ICD operating in a manual mode by a patient provided with a patient activator; and

FIGS. 5A and 5B are a flow chart illustrating the method of the present invention practiced in an atrial ICD operating in either an automatic mode or in a manual mode by a patient provided with a patient activator.

### DETAILED DESCRIPTION OF THE INVENTION

In the following detailed description, references are made to illustrative embodiments of methods and apparatus for carrying out the invention. It is understood that other embodiments can be utilized without departing from the scope of the invention. Preferred methods and apparatus are described for accessing the pericardial space between the epicardium and the pericardium as an example of accessing an anatomic space between an outer tissue layer and an inner tissue layer.

The present invention is preferably implemented in the context of an atrial ICD having AT/AF detection and atrial C/D therapy delivery capabilities. The illustrated preferred embodiment of the invention is implemented in an atrial and ventricular ICD of the type described above with reference to the above-referenced MEDTRONIC® GEM III AT Model 7276 IPG, for example.

Such dual chamber ICDs may be constructed or made programmable to provide atrial only, ventricular only, or both atrial and ventricular pacing modes during bradycardia episodes and to provide AT/AF detection and therapy delivery, particularly AF C/D shock therapies, as well as backup VT/VF detection and therapy delivery. In addition, the present invention may be employed with a wide variety of pacing and C/D electrode combinations as described in commonly assigned U.S. Pat. Nos. 5,165,403, 5,292,338, and 6,330,477, for example.

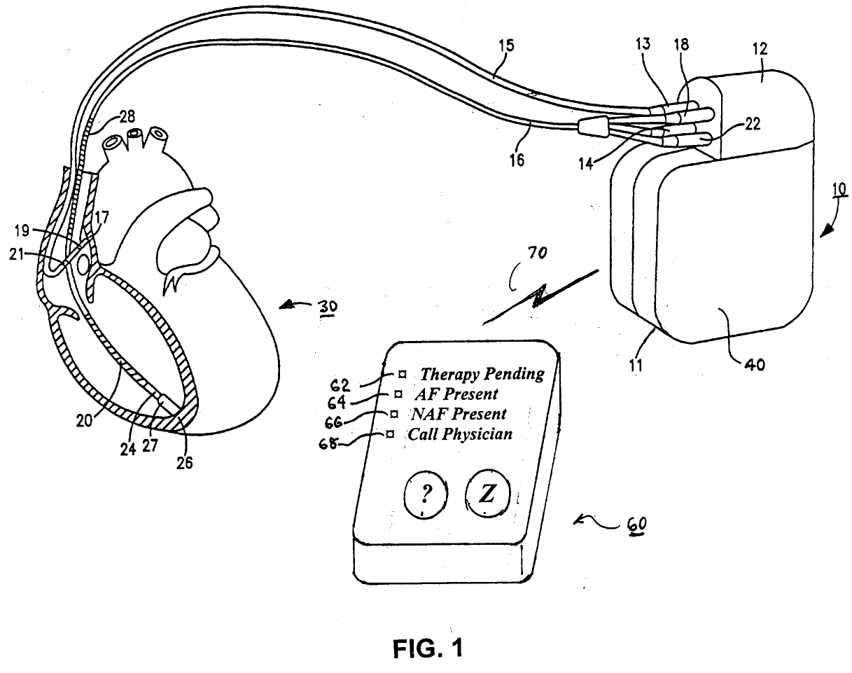

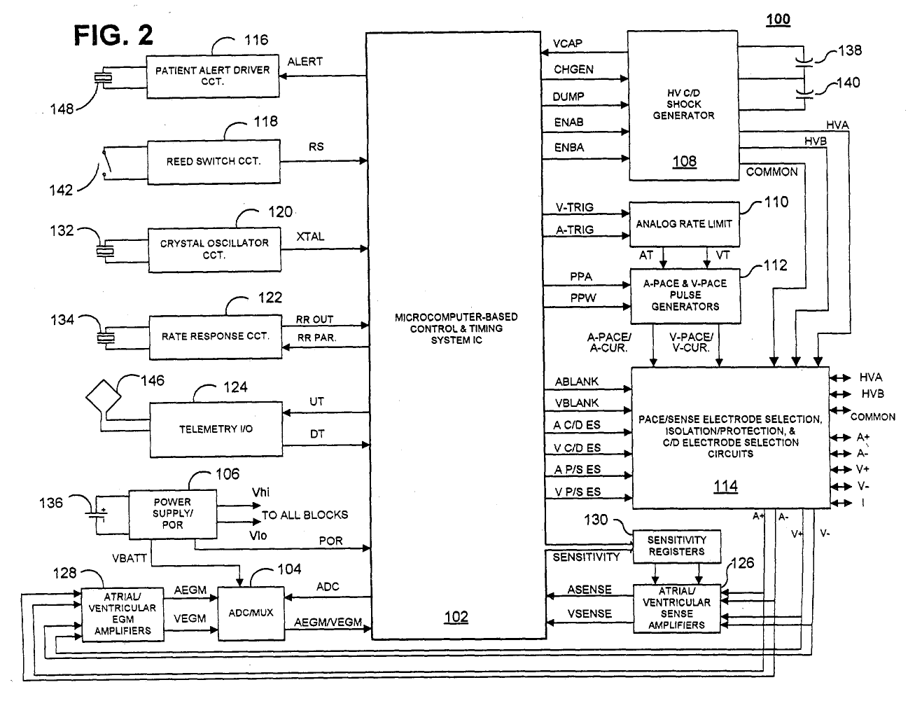

FIGS. 1 and 2 illustrate such a dual chamber, multi-programmable, ICD IPG and associated lead system for providing atrial and/or ventricular sensing functions for detecting P-waves of atrial depolarizations and/or R-waves of ventricular depolarizations, depending on the programmed pacing and/or sensing mode and delivering pacing and/or C/D therapies. FIGS. 1 and 2 are intended to provide a comprehensive illustration of each of the atrial and/or ventricular, pacing and/or C/D configurations that may be effected using sub-combinations of the components depicted therein and equivalents thereto. Preferably, bipolar pace/sense electrodes are employed in the practice of the invention, but their configuration, fixation in contact with, and positioning with respect to the atria and ventricles may differ from those shown in FIG. 1. Unipolar pace/sense electrode bearing leads may also be used in the practice of the invention, and the second, return electrode may be one or more of the C/D electrodes.

FIG. 1 illustrates an ICD IPG 10 and a lead set comprising leads 15 and 16 extending to the atrial and ventricular chambers of heart 30 that can be employed in the practice of the present invention. The ICD IPG is formed of a hermetically sealed housing 11 enclosing the electronic circuitry and battery of the ICD system and a connector block 12 containing electrical connectors for coupling the various connector rings and pins of the proximal lead connectors 13, 14, 18 and 22 of the leads 15 and 16. Optionally, insulation of the outward facing portion of the housing 11 of the ICD IPG 10 may be provided in the form of a plastic coating, for example parylene or silicone rubber, as is currently employed in some unipolar cardiac pacemakers. However, the outward facing portion may instead be left un-insulated, or some other division between insulated and un-insulated portions may be employed. The un-insulated portion of the housing 11 can then serve as a subcutaneous CAN C/D electrode 40.

The ventricular lead 16 includes an elongated, non-conductive, bio-compatible lead body enclosing four mutually insulated lead conductors. A ring shaped pace/sense electrode 24, an extendable helix pace/sense electrode 26, mounted retractably within an non-conductive electrode head 27, an elongated, wire coil, RV C/D electrode 20, and an elongated, wire coil, SVC C/D electrode 28 are located on lead body 16 and coupled to distal ends of one of the coiled conductors within the lead body 16. Ventricular pace/sense electrodes 24 and 26 are employed for applying cardiac pacing pulses to the ventricle and for sensing ventricular depolarizations or R-waves. Ventricular lead 16 is formed with three bifurcated proximal connectors 14, 18 and 22 coupled with the proximal ends of the lead conductors within the lead body. Unipolar connectors 18 and 22 each terminate in connector pins coupled to the proximal ends of the lead conductors coupled with the coiled wire, C/D electrodes 20 and 28, respectively. Proximal connector 14 is an in-line bipolar connector carrying a connector ring and a connector pin that are coupled through lead conductors to ventricular pace/sense electrodes 24 and 26, respectively.

The conventional bipolar atrial pacing lead 15 includes an elongated non-conductive lead body enclosing two concentric coiled wire lead conductors that are separated from one another by tubular non-conductive sheaths and extend from the proximal connector 13 to atrial pace/sense electrodes 21 and 17 disposed in the right atrium. The ring-shaped, atrial pace/sense electrode 21 and an extendable helix, atrial pace/sense electrode 17, mounted retractably within an non-conductive electrode head 19, are located adjacent the J-shaped distal end of the atrial pacing lead body and are employed for atrial pacing and for sensing atrial depolarizations or P-waves. In alternative atrial lead systems, a C/D electrode, for example corresponding to SVC C/D electrode 28, might instead be mounted to the atrial lead 15. A further CS C/D lead could also be employed in the ICD system for location in the coronary sinus and great cardiac vein as also shown in FIG. 1 of the above-referenced '834 patent, for example.

The ICD IPG 10 can be programmed to select an atrial C/D shock delivery pathway between the RV C/D electrode 20 and the CAN C/D electrode 40+the SVC C/D electrode 28 as described above in response to detection of an AF episode or in response to an AF therapy delivery command received from the patient. The ICD IPG 10 can be programmed to select a ventricular C/D shock delivery pathway between the RV C/D electrode 20 and the CAN C/D electrode 40, for example, for delivery of a ventricular C/D shock in response to detection of a VF episode. It should be noted that the ventricular C/D shock delivery pathway could be selected to be the same as the atrial C/D shock delivery pathway. The primary difference in delivery is simply the greater magnitude of the ventricular C/D shock than the magnitude of the atrial C/D shock. A patient activator 60 of the type disclosed in the above-referenced '857 patent and preferably corresponding to the MEDTRONIC® InCheck™ patient activator is also depicted in FIG. 1. The patient activator 60 communicates commands to and receives data from the ICD IPG 10 through downlink telemetry (DT) and uplink telemetry (UT) transmissions when a telemetry link 70 through the patient's skin is established between telemetry antennae and telemetry transceivers in the patient activator 60 and the ICD IPG 10 in a manner well known in the art. The patient activator includes labeled LEDs 62, 64, 66 and 68, a query button marked “?”, and a therapy delivery command button marked with a shock symbol “Z”. The patient can depress the “?” button to DT transmit a status query, and the ICD IPG responds by UT transmitting status data indicating one of “AF Present”, “NAF (no AF) Present” as determined by the AF detection algorithm. “AF Present” indicates that AF is present, but the patient requested atrial C/D shock therapy cannot be delivered for safety reasons because the detected R-R interval is consistently shorter than a programmed minimum R-R interval. “No AF Present” indicates that an AF episode is not detected by the AF detection algorithm and that the patient requested atrial C/D shock therapy will not be delivered. The message “Therapy Pending” is highlighted after the patient has depressed the shock button and indicates that the high voltage output capacitors are charging to deliver the programmed patient-activated therapy. The “Call Physician” message is highlighted if no programmed therapies remain to be delivered or the ICD does not have patient activated therapies programmed on. FIG. 2 is a functional schematic diagram of an ICD IPG operating system 100 within the IPG housing 11 and with which the present invention may usefully be practiced. This diagram should be taken as exemplary of the type of ICD system 100 in which the invention may be embodied, and not as limiting, as it is believed that the invention may usefully be practiced in a wide variety of device implementations.

The ICD IPG 10 preferably comprises an ICD operating system that provides the operating modes and functions of the MEDTRONIC® GEM® III AT Model 7276 IPG that is programmable in operating mode and parameter values and interrogatable employing the MEDTRONIC® Model 9790C external programmer (not shown), for example. The programming of ICD operating modes and parameters or the interrogation of data stored in the ICD IPG 10 or the initiation of UT transmission of the cardiac EGM is accomplished or initiated via programming or interrogation commands transmitted in a DT transmission by the programmer to an ICD telemetry antenna. The ICD IPG telemetry system decodes the commands in the DT transmission and either forwards programming instructions to the ICD operating system or formats the requested data or cardiac EGM retrieved by the operating system and UT transmits it to the external programmer.

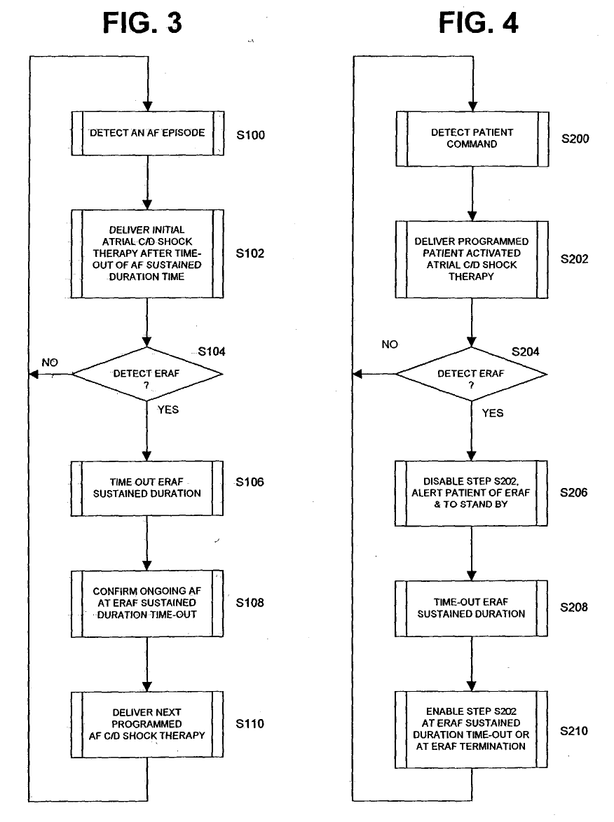

FIG. 2 is a functional block diagram illustrating such a dual chamber ICD operating system 100 that is merely exemplary of a variety of single chamber and dual chamber ICD systems having all or some of the capabilities described herein in which the operations of the present invention enabling detection of ERAF and delaying delivery of a second atrial C/D shock can be advantageously implemented. In this regard, it should be understood that the following description of a suitable ICD operating system 100 sets forth a number of features and operating modes that may or may not be necessarily employed in the practice of the present invention. The present invention can be practiced in the context of automatic detection of AF and automatic delivery of an atrial C/D shock or patient perception of AF and patient initiated delivery of atrial C/D shock, the detection of ERAF and delaying delivery of a second atrial C/D shock to avoid delivering an ineffectual atrial C/D shock. The delivery of an atrial C/D shock presents the risk of inducing a ventricular tachyarrhythmia, e.g., VF. Consequently, a dual chamber ICD operating system 100 is described that can function to deliver a range of appropriate ventricular anti-tachyarrhythmia therapies if a ventricular tachyarrhythmia is induced or spontaneously occurs. The ICD system 100 operates employing atrial and ventricular tachyarrhythmia detection and discrimination algorithms, and also incorporates atrial and ventricular dual chamber bradycardia and anti-tachycardia pacing capabilities. Similar ICD systems to that depicted in FIG. 2 in which the present invention can be implemented are shown, for example, in the above-referenced '834 patent, for example. The exemplary ICD system 100 is first described as a whole and the particular operating modes of the present invention are then described in reference to FIGS. 3-5.

The ICD system 100 includes one of more ICs typically mounted on one or more hybrid circuit, a PC board mounting a number of discrete components, and further large scale, discrete components. The heart of the ICD operating system is in hardware and software in a microcomputer-based timing and control system IC 102 that is coupled with the other system blocks of ICD system 100. Various depicted signal and control lines interconnecting these blocks, but not all are shown for simplicity of illustration and because they play no material role in the practice of the present invention.

The large scale, discrete, off-board, components include one or more batteries 136, HV output capacitors 138, 140, and housing mounted, patient alert sound transducer 148 and a patient activity sensor 134. The discrete components mounted to the PC board include telemetry antenna 146, reed switch 142, crystal 132, a set of HV discrete components of the HV C/D shock generator 108, and switching and protection circuit components of block 114. These discrete components are coupled to system IC 102 through other ICs and hybrid circuits incorporating the functional blocks 104-130 described further below. The depicted functional blocks and discrete components of FIG. 2 can be arranged as one or two LV hybrid circuits, a HV hybrid circuit, and a discrete component PC board. However, it will be understood that a single hybrid circuit could be employed that incorporates and supports all of the system ICs.

The exemplary ICD operating system 100 of FIG. 2 is powered by the battery 136 coupled to power supplies in power source block 106 for developing regulated high and low voltage power supplies Vhi and Vlo that are supplied to selected ones of the other functional blocks. Preferably, battery 136 is a lithium SVO battery that can be employed to provide HV capacitor charging current and that produces a voltage from about 3.2 volts when fresh to about 2.5 volts at specified end of service for a single chamber ICD and twice these values for a dual chamber ICD. Power supply 106 also includes a power-on-reset (POR) circuit that generates a POR signal initially when the battery 136 is connected with power supply 106 and any time that the voltage of battery 136 falls below a threshold voltage.

The crystal oscillator circuit 120 is coupled to clock crystal 132 and provides one or more system XTAL clock that is applied to the microcomputer-based control and timing system IC and distributed to other blocks of FIG. 2 as appropriate.

The telemetry I/O circuit 124 includes an UT transmitter and a DT receiver coupled with the IPG telemetry antenna 146. The UT transmitter of telemetry I/O circuit 124 receives formatted UT data from telemetry I/O registers and control circuitry in system IC 102 for UT transmission to the antenna and transceiver in the external programmer-or in the external patient activator 60. The DT receiver of telemetry transceiver circuit 124 receives DT signals transmitted from the external programmer or in the external patient activator 60 and decodes and forwards the DT signals to the telemetry I/O registers and control circuitry in system IC 102.

The telemetry I/O circuit 124 is enabled to receive and decode DT transmitted interrogation and programming commands from the external programmer when the reed switch circuit 118 provides the RS signal upon closure of reed switch 142 by an external programming head magnetic field. DT transmitted RF signals ring an L-C tank circuit including the IPG telemetry antenna 146. Other IMD functions are also affected when a magnetic field closes the reed switch 142 and the RS signal is generated in a manner well known in the art.

The rate response circuit 122 is coupled to a physiologic activity sensor 134, which is preferably a transducer or accelerometer mounted to the IPG housing 11 inner surface, and provides activity correlated RR output signals to the rate response circuit 122 in a manner well known in the art. The RR output signal processing by the rate response circuit 122 can be programmed by RR PAR instructions and parameter values in a number of ways known in the art.

A patient alert driver circuit 116 is illustrated in FIG. 2 coupled to an audible sound emitting transducer 148 that is mounted adjacent to the interior surface of the IPG housing 11 and is powered by ALERT signals to emit audible warning signals in high urgency and low urgency tones to alert the patient of events or conditions of concern warranting physician intervention. The patient alert driver circuit is not presently implemented in the above-referenced MEDTRONIC® GEM III AT Model 7276 IPG.

The operating modes of the ICD operating system 100 are controlled by the system IC 102 depicted in FIG. 2 that receives the depicted signals from and provides signals and data to the blocks 104-128 and 130 of FIG. 2. The system IC 102 comprises the typical components of a microcomputer including a microprocessor, a CPU, RAM, ROM, and further operating system control circuitry that is conveniently located with it. The operating modes and parameters are programmable and interrogatable through DT programming and interrogation operations via the telemetry I/O registers. Such operating modes include a number of pacing, monitoring, tachyarrhythmia detection and therapy delivery modes and such operating parameters include pacing pulse width and/or amplitude, sense amplifier sensitivity, event data storage, atrial and ventricular tachyarrhythmia detection parameters in a given mode, the tachyarrhythmia therapy parameters, etc.

The electrode selection block 114 provides a number of functions in response to commands of the system IC 100 that select, sensing, pacing and C/D pathways among the available electrodes, provide blanking of sense amplifiers and otherwise protect circuitry from being affected by C/D shocks that are delivered.

The depicted HV C/D shock generator 108 comprises a DC-DC converter and a HV output or discharge circuit for discharging the charge on the HV output capacitor bank 138 and 140 through the programmed atrial and ventricular C/D pathways. The DC-DC converter comprises a HV charging circuit, a discrete HV step-up transformer, and the HV output capacitor bank 138 and 140 coupled to the secondary transformer coils. The charge on the HV output capacitor bank 138 and 140, in this case, is selectively discharged through selective connection of the terminals HVA, HVB and COMMON to the lead conductors coupled to with the RV C/D electrode 20 and the SVC C/D electrode 28 and/or to the CAN C/D electrode 40 of FIG. 1 via HV switches in the switch circuit block 114. The HV switches defining the atrial and ventricular C/D pathways are closed by the respective atrial and ventricular C/D electrode select signals (A C/D ES and V C/D ES). The HV C/D shock generator 108 can also be commanded to test charge or reform the HV output capacitors 138, 140 or to provide a C/D lead impedance test current.

The output capacitors 138 and 140 are coupled to secondary windings of a step-up transformer by means of diodes of HV C/D shock generator 108. The primary winding of the step-up transformer is coupled to HV charging circuitry, and the HV output capacitors 138 and 140 are charged to a programmed C/D shock energy in a manner well known in the art. The HV charging circuitry is controlled by the CHDR signal supplied by control circuitry within system IC 102 when a malignant atrial or ventricular tachyarrhythmia subject to C/D therapy is detected. The voltage across the output capacitors 138 and 140 is monitored and applied via the VCAP signal to C/D HV charge control circuitry within the system IC 102 that detects the point when the VCAP signal level matches the programmed energy level of the C/D shock to be delivered and terminates the CHDR signal. The C/D HV charge control circuit within the system IC 102 provides the first control signal ENAB, the second control signal ENBA, and the DUMP signal that initiate discharge of the charge stored across the output capacitors 138 and 140 to deliver the programmed monophasic or biphasic C/D shock to the selected C/D electrodes.

The atrial and ventricular sense amplifiers in block 126 are coupled to the A+, A− and V+, V− pacing lead conductors through pace/sense selection and protection circuitry within electrode selection block 114 in response to the respective atrial pace/sense electrode selection (ASP ES) and ventricular pace/sense electrode selection (VSP ES) signals. The atrial and ventricular sense amplifiers in block 126 each comprise a programmable gain, bandpass amplifier, a threshold setting circuit and a comparator for comparing the bandpass filtered atrial and ventricular cardiac signal amplitude to the threshold. The atrial and ventricular sensing thresholds of the respective atrial and ventricular sense amplifiers are programmable, and the programmed SENSITIVITY signals are applied to the sensitivity registers 130. The atrial and ventricular sense amplifiers generate ASENSE and VSENSE signals, respectively, when their inputs are not blanked by blanking circuits in block 114 that respond to the ABLANK and VBLANK signals and the atrial and ventricular cardiac signal amplitudes exceed the respective atrial and ventricular sensing thresholds.

The atrial and ventricular cardiac signals conducted through the A+, A− and V+, V− pacing leads are also applied to inputs of atrial and ventricular EGM amplifiers 128 that supply the amplified AEGM and VEGM signals to ADC/MUX 104. In the ADC/MUX 104, the AEGM and VEGM are continually sampled and digitized, and the digitized AEGM and VEGM are provided to RAM memory registers or buffers in system IC 102 for temporary storage on a FIFO basis. When an atrial or ventricular tachyarrhythmia episode is detected, the temporarily stored, digitized AEGM and VEGM values are shifted into memory registers. Then, succeeding digitized AEGM and VEGM values are also stored in memory registers to provide programmable length AEGM and VEGM strips preceding and following the detection of the arrhythmia and encompassing the delivery of the tachyarrhythmia therapies.

Data related to the delivered therapies is also stored in association with the AEGM and VEGM strips. Due to memory limitations, the stored AEGM and VEGM strip may be discarded and replaced each time a tachyarrhythmia is detected. However, historic episode logs are compiled and incremented in RAM in system IC 102 that provide the date, time, type of episode, cycle length, duration, and identify the last stored EGM strip. Other episode records stored in registers in RAM in system IC 102 are also maintained related to V-V intervals, numbers of single premature ventricular contractions (PVCs), runs of PVCs, tachyarrhythmias detected, therapies delivered, successful and unsuccessful therapies, etc.

Dual chamber pace/sense timing and control circuitry within the system IC 102 provides inputs to and carries out many of the operations governing the atrial and ventricular pacing modes of operation, which are preferably programmable. The rate response signal RR, the reed switch signal RS, the XTAL clock signal, and the ASENSE and VSENSE signals generated in blocks of FIG. 2 are applied to the pace/sense timing and control circuitry. The pace/sense timing and control circuitry times out synchronized atrial and ventricular pacing escape intervals that depend on the current operating mode and programmed lower and upper pacing rates. The atrial and ventricular pacing rates are allowed to vary between the lower and upper pacing rates in dependence upon the RR signal when physiologic pacing modes e.g., DDDR and DDIR modes, are programmed and not otherwise inhibited by excessive atrial heart rates.

In these operating modes, when the respective programmed escape interval timers time out, the pace/sense timing and control circuitry within the system IC 102 generates the A-TRIG and/or V-TRIG pacing trigger signals that are applied to the analog rate limit circuit 110. The analog rate limit circuit 110 generates the AT and VT signals applied to the A-PACE and V-PACE pulse generators in pacing output block 112 of FIG. 2 after the atrial and ventricular rate limit timers time out in order to limit the atrial and ventricular pacing rates to safe upper pacing rates. The A-PACE and V-PACE pulse generators in pacing output block 112 respond to the AT and VT signals, respectively to generate the respective A-PACE and V-PACE pulses at pace pulse energies, that is, either or both of the pacing pulse width and amplitude. The pace pulse energies that are programmed by the physician and stored in RAM 152 and are applied as PPA and PPW signals from pace/sense timing and control circuitry within system IC 102 to the A-PACE and V-PACE pulse generators in pacing output block 112.

The pace/sense timing and control circuitry within system IC 102 also generates the A-BLANK blanking signals having programmed blanking intervals following any of the ASENSE, VSENSE, A-TRIG and V-TRIG signals and the V-BLANK blanking signals having programmed blanking intervals following both VSENSE and V-TRIG signals. The A-BLANK and V-BLANK signals are applied to the pace/sense isolation circuits in block 114 of FIG. 2 for rendering the atrial and ventricular sense amplifiers in block 126 of FIG. 2 inoperative for these programmed blanking intervals. Atrial and ventricular sense amplifier refractory periods are also timed out from these signals in the pace/sense timing and control 170 and employed to avoid resetting the respective atrial and ventricular escape intervals timed out therein if an ASENSE or VSENSE event falls within a respective refractory period to avoid inappropriate responses in a manner well known in the art.

The pace/sense timing and control circuitry also implements certain operations of the tachyarrhythmia detection and classification algorithms carried out by the microcomputer and stored in ROM and RAM in the system IC 102. The intervals between and rates of ASENSE and VSENSE signals are tracked and compared to rate thresholds and other detection criteria to determine the onset of AT or AF episodes and VT or VF episodes, respectively. The tachyarrhythmia detection criteria typically involves elevation of the spontaneous heart rate coupled with other onset, rate acceleration, stability criteria and various other criteria, e.g., morphology, for example. The tachyarrhythmia detection criteria are specified in ROM, and programmable detection parameters are stored in RAM. The spontaneous heart rate is calculated in a heart rate timer maintained by the microcomputer 130, and other characteristics of the EGM are examined to determine whether or not a high rate EGM constitutes a normal sinus rhythm or a malignant tachyarrhythmia. Spontaneous heart rate and EGM width criterion are employed in the MEDTRONIC® GEM® 7227 single chamber ICD IPG, and both the atrial and ventricular heart rates and EGMs are examined with information about conduction patterns, regularity and AV dissociation in the detection and classification algorithm employed in MEDTRONIC® GEM® DR 7271 dual chamber ICD and the MEDTRONIC® GEM III AT Model 7276 IPG IPGs. Suitable atrial and ventricular tachyarrhythmia detection and discrimination methods and algorithms are set forth in commonly assigned U.S. Pat. No. 5,991,656, for example

When a tachyarrhythmia episode is detected and classified, the appropriate programmed burst-pacing therapy or synchronous cardioversion shock therapy or defibrillation shock therapy is delivered. A group of successively more aggressive therapies can be programmed to be delivered to convert a specified atrial or ventricular tachyarrhythmia that persists despite the delivery of the initial therapies of the group. If an atrial or ventricular tachycardia is detected by an atrial or ventricular tachycardia detection algorithm embodied in the microcomputer, and if an anti-tachycardia pacing therapy is programmed to treat it, then the pace/sense timing and control circuit is employed to trigger delivery of high rate A-PACE or V-PACE pacing pulses via A-PACE and V-PACE pulse generators in pacing output block 112. Again, the pacing pulse energy of each pacing pulse and the blanking periods following each pacing pulse are programmable. A number of other characteristics of high rate pacing pulse therapies are programmable establishing a fixed or adaptive (ramp) pacing rate, the number of pacing pulses in the burst, and the number of times that the burst can be supplied to terminate a single tachycardia episode.

The C/D shock therapies are delivered through therapy delivery commands applied to the HV C/D shock generator 108, which responds in the manner described above. Additionally, pacing regimens can also be programmed and timed out in the pace/sense timing and control circuitry and generated in A-PACE and V-PACE pulse generators in pacing output block 112 for pacing for a programmed time following delivery of a C/D shock therapy to capture the heart and establish a normal sinus rhythm.

The ASENSE and VSENSE events are analyzed following delivery of a C/D shock to determine whether the atrial or ventricular tachyarrhythmia has terminated. In regard to AF termination, typically, an AF episode is declared terminated if five consecutive P-waves are detected at an atrial heart rate that is lower than an AT or AF detection threshold rate. For example, the AF episode is declared terminated and the delivered atrial C/D shock therapy is considered “successful” if five such “sinus beats” are detected within three minutes of the delivery of the atrial C/D shock.

If these conditions are not satisfied, the delivered atrial C/D shock therapy is considered “unsuccessful” and AF is not declared terminated. In effect, the AF detection criteria are again met, and a second atrial C/D shock is delivered, if programmed, after time-out of an AF sustained duration. However, delivery of a second atrial C/D shock is not recommended in many cases or cannot be delivered if a daily availability period has expired.

As also described above, an ERAF often occurs shortly following the delivery of an initial atrial C/D shock and declaration that the AF episode is declared terminated by the delivered atrial C/D shock. The ICD operating system 100 can be programmed to allow more than one atrial C/D shock in a given period, e.g., the 24 hour AF therapy delay, and the AF detection algorithm will detect the ERAF as a new AF episode and trigger delivery of another atrial C/D shock. As noted above, the early recurring AF episode will likely recur again following delivery of the second atrial C/D shock. Therefore, it would be preferable to allow more than one atrial C/D shock to be delivered but to delay that delivery for an ERAF sustained duration that has been shown to increase the possibility that the applied second atrial C/D shock will not be followed by ERAF.

Therefore, FIG. 3 is a simplified depiction of the automatic mode of detecting and responding to AF episodes employing the dual chamber ICD IPG 10 of FIGS. 1 and 2 to detect ERAF (step S104) following delivery of an initial atrial C/D shock (step S102) and to delay delivery of a second atrial C/D shock (step S110) until the programmed ERAF sustained duration times out (step S108). Therefore, the delivery of the second atrial C/D shock in step S110 after the programmed ERAF sustained duration times out in step S108 and AF detection criteria are still met. In this way, the likelihood that the second atrial C/D shock delivered in step S110 will be successful is increased.

The algorithm of FIG. 3 assumes that at least an initial and a second atrial C/D shock are programmed to be delivered in any given daily availability period and that the daily availability period is longer than the ERAF sustained duration to permit delivery of the second atrial C/D shock in step S110. It is presumed in this embodiment, that the physician has programmed the ICD IPG 10 to detect and/or respond to AF episodes only after lapse of an AF therapy delivery delay, e.g., 24 hours, since delivery of a preceding AF C/D shock. It will be understood that the expiration of the daily availability period can prevent the delivery of the second atrial C/D shock in step S110 if the daily availability period terminates before the programmed ERAF sustained duration times out in step S108. It should also be noted that the initial delivery of the initial atrial C/D shock can be delayed in step S102 if an AF sustained duration of minutes to hours is programmed. In other words, the AF episode must continue for a programmed AF sustained duration since it is first detected before an initial atrial C/D shock can be delivered in step 102. Similarly, the ERAF episode must continue for a programmed ERAF sustained duration since it is detected in step S104 before the second atrial C/D shock can be delivered in step S110.

Ignoring the potential interference with termination of the daily availability period, it is assumed that the ICD IPG 10 is programmed by the physician to deliver a second atrial C/D shock in step S110 if delivery of an initial atrial C/D shock in step S102 is followed by detection of ERAF. After the initial atrial C/D shock is delivered in step S102, ERAF can occur as determined in step S104, and time-out of an ERAF sustained duration is commenced in step S106 that inhibits delivery of the second atrial C/D shock until the ERAF sustained duration times out and continued AF is confirmed in step S108. Step S108 is not satisfied if ERAF fails to be sustained over the full ERAF sustained duration. In other words, if the ERAF episode spontaneously terminates during time-out of the ERAF sustained duration, then step S108 and S110 are not followed. Any AF episode that is detected in step S100 following determination that ERAF has spontaneously terminated is treated as a new AF episode, and delivery of a new initial atrial C/D shock may be prohibited in step S102, depending on the programmed number of initial atrial C/D shocks that can be delivered in any 24 hour period, for example.

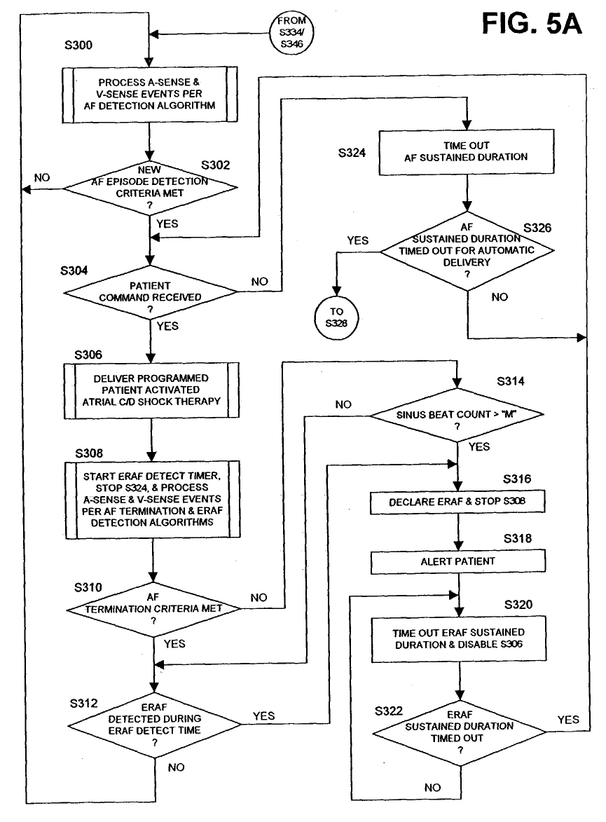

Turning to FIG. 4, when the patient initiates delivery of an atrial C/D shock, and that delivered atrial C/D shock is followed by ERAF, the patient again feels the AF symptoms and concludes that the initial atrial C/D shock was unsuccessful. The patient then may initiate delivery a second atrial C/D shock into the ERAF that is again “unsuccessful”. The algorithm of FIG. 4 inhibits the delivery of a second atrial C/D shock into an ERAF until an ERAF sustained duration times out or the ERAF episode spontaneously terminates. In this way, the likelihood that the second delivered atrial C/D shock will be successful is increased. If the ERAF spontaneously terminates then the patient will know this when they press the query button on the activator 60 (yellow light does not go on). Pressing the therapy button of the activator 60 will only cause a shock to be delivered if AF is determined to be present by the AF detection algorithm as noted above.

In FIG. 4, it is presumed that the automatic delivery of an atrial C/D shock is programmed OFF or is not present in the atrial ICD. The patient is provided with a patient activator 60, and the AF sustained duration time is set to a minimal time or is programmed OFF. The patient perceives AF symptoms and uses the patient activator 60 of FIG. 1 to DT transmit a patient command that is received and detected by the ICD operating system 100 to trigger delivery of a programmed atrial C/D shock in step S200. It is presumed that the patient may be inclined to initiate delivery of a second atrial C/D shock if the patient perceives that first atrial C/D shock delivered in step S202 has failed to cardiovert the AF. In this instance, if ERAF is detected in step S204, step S202 is disabled in step S206 until time-out of an ERAF sustained duration in step S208. Optionally, the patient is advised that the first delivered atrial C/D shock has been followed by a closely timed ERAF in step S206, so that the patient can then refrain from attempting to initiate the delivery of the further atrial C/D shock. In step S210, the delivery of a further atrial AF shock in step S202 in response to a further detected patient command is enabled after time-out of the ERAF sustained duration or termination of the ERAF episode is detected. In this way, any attempt to initiate delivery in step S200 is ignored in step S202 until the ERAF sustained duration is timed out in step S208 or the ERAF episode spontaneously terminates.

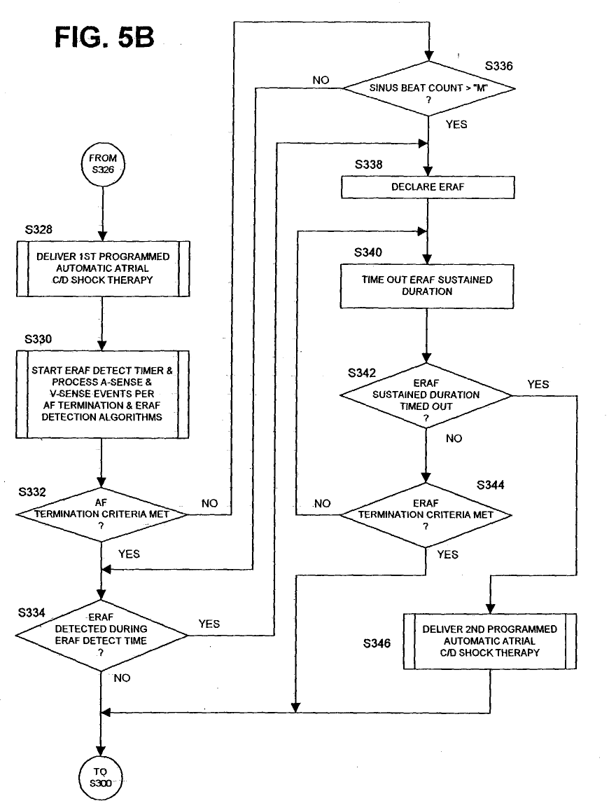

FIGS. 5A and 5B depict the steps of a more detailed flow chart of a preferred response to ERAF by an ICD 10 that can operate both automatically or in response to a patient initiated command using the patient activator 60. In step S300, the A-SENSE and V-SENSE events output by the atrial and ventricular sense amplifiers are processed in accordance with the AF detection algorithm. In this embodiment, the physician can define new AF episode detection criteria that include only those occurring after a programmed AF therapy delay in step S300. When step S302 is satisfied, the AF sustained duration is timed out in step S326. If the AF sustained duration time times out as determined in step S326, then the steps S328-S344 of FIG. 5B are followed to deliver a atrial C/D shock therapy and respond to any ERAF following delivery of the AF shock that continues for the full ERAF sustained duration. However, the patient can also feel AF symptoms due to the AF episode being detected in step S300-S302 and respond by using the patient activator 60 as described above to DT transmit a patient command that is received and detected in step S304. Steps 306-S322 are followed if the patient command is received in step S304 before time-out of the AF sustained duration is detected in step S326.

The programmed atrial C/D shock is delivered in step S304 if a patient command is received in step S304. In step S308, the time-out of the AF sustained duration in step S324 is halted, time-out of an ERAF detect time is started, and post-delivery A-SENSE and V-SENSE events are examined to determine if AF termination criteria are met. The AF termination criteria may be met as determined in step S310 by the detection of a certain number “N” of sinus A-SENSE events in relation to V-SENSE events, (e.g., 5 sinus beats) within a termination time window.

In one aspect of the invention, ERAF may be the cause of the failure to meet the AF termination criteria in step S312, rather than simply a failure of the delivered atrial C/D shock to terminate the AF episode. ERAF may be defined as the resumption of AF if a programmed sinus beat count “M” (e.g., 2-4 sinus beats) that is less than the AF termination sinus beat count is satisfied. In other words, if M of N sinus beat counts are determined in step S314, then step S308 is terminated and ERAF is declared in step S316.

In a further aspect of the invention, assuming that step S314 is not satisfied, the A-SENSE and V-SENSE events continue to be processed during a programmable ERAF detect time or sinus beat count that exceeds the AF termination sinus beat count in step S308. For example, the ERAF detect time can be programmed as one minute to six hours. If ERAF detection criteria are not satisfied in step S314, then the AF episode is effectively terminated, and the step S300 is followed. However, if ERAF detection criteria are satisfied in step S312, then step S308 is terminated and ERAF is declared in step S316.

The patient is alerted of the ERAF declaration in step S318 and the time-out of the ERAF sustained duration is started in step S320 if ERAF is declared in step S316 in satisfaction of either of steps S312 or S314. Step S306 is disabled for the duration of the ERAF sustained duration that is timed out in step S320 and is enabled after time-out of the ERAF sustained duration as determined in step S324.

Thus, if ERAF is determined to have occurred, the patient is prevented from triggering delivery of a further atrial C/D shock until the ERAF sustained duration times out.

Turning to the automatic mode of steps S328-S346 of FIG. 5B, the first programmed automatic atrial C/D shock therapy is delivered in step S328 once the AF sustained duration times out in step S324 as determined in step S326 of FIG. 5A. Then, steps S330-S342 track steps S308-S322 as described above, except that the patient is not alerted to the ERAF detection per step S318 of FIG. 5A, because the patient may be asleep at the time that step S302 is satisfied. In addition, the second programmed automatic atrial C/D shock therapy is delivered in step S346 only if the ERAF episode does not spontaneously terminate as determined in step S344 while the ERAF sustained duration times out in step S340. It should be noted that the delivery of a second programmed automatic atrial C/D shock therapy in step S346 may be inhibited if the daily availability period (if programmed) expires before time-out of the ERAF sustained duration time as determined in step S342.

Thus, the automatic delivery of a second programmed automatic atrial C/D shock therapy is delayed if ERAF is determined after delivery of the first atrial C/D shock in any given 24 hour period, for example, and is only delivered if the ERAF episode continues throughout the ERAF sustained duration.

It will be appreciated that the algorithms of FIGS. 3, 4 and 5A-5B can be practiced in the context of a wide variety of atrial ICDs.

It will be understood that certain of the above-described structures, functions and operations of the above-described preferred embodiments are not necessary to practice the present invention and are included in the description simply for completeness of an exemplary embodiment or embodiments.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

What is claimed is:

1. A method of decreasing incidences of early recurrence of atrial fibrillation (ERAF) following delivery of an atrial cardioversion/defibrillation (C/D) shock to the atria of a patient's heart by an implantable cardioverter/defibrillator comprising:
detecting ERAF, if present, following delivery of an atrial C/D shock;
timing out an ERAF sustained duration upon detecting ERAF during the ERAF detect time; and
inhibiting delivery of a further atrial C/D shock for the duration of the ERAF sustained duration.

2. The method of claim 1, further comprising:
determining if atrial fibrillation (AF) is present upon time out of the ERAF sustained duration; and
delivering a further atrial C/D shock to the atria upon determining that AF is present upon time out of the ERAF sustained duration.

3. The method of claim 2, further comprising:
detecting AF termination during time out of the ERAF sustained duration; and
terminating the ERAF sustained duration in response to detected AF termination.

4. The method of claim 1, further comprising:
detecting AF termination during time out of the ERAF sustained duration; and
terminating the ERAF sustained duration in response to detected AF termination.

5. A method of decreasing incidences of early recurrence of atrial fibrillation (ERAF) following delivery of an atrial cardioversion/defibrillation (C/D) shock to a patient's heart by an implantable cardioverter/defibrillator responsive to a patient command generated by a patient activator operated by a patient experiencing symptoms of an atrial fibrillation (AF) episode comprising:
delivering the atrial C/D shock to the patient's heart in response to the patient command;
detecting ERAF, if present, following delivery of an atrial C/D shock;
timing out an ERAF sustained duration upon detecting ERAF during the ERAF detect time; and
inhibiting delivery of a further atrial C/D shock in response to a further patient command for the duration of the ERAF sustained duration.

6. The method of claim 5, further comprising alerting the patient that ERAF is detected and that delivery of the further atrial C/D commanded by the patient is inhibited.

7. A method of decreasing incidences of early recurrence of atrial fibrillation (ERAF) following delivery of an atrial cardioversion/defibrillation (C/D) shock to a patient's heart by an implantable cardioverter/defibrillator responsive to a an atrial fibrillation (AF) episode comprising:
detecting an AF episode;
delivering the atrial C/D shock to the atria of the patient's heart in response to the detected AF episode;
detecting ERAF, if present, following delivery of the atrial C/D shock;
timing out an ERAF sustained duration upon detecting ERAF during the ERAF detect time; and
delivering a further atrial C/D shock upon time out of the ERAF sustained duration.

8. The method of claim 7, further comprising:
determining if atrial fibrillation (AF) is present upon time out of the ERAF sustained duration; and
delivering the further atrial C/D shock to the atria only upon determining that AF is present upon time out of the ERAF sustained duration.

9. The method of claim 8, further comprising:
detecting AF termination during time out of the ERAF sustained duration; and
terminating the ERAF sustained duration in response to detected AF termination.

10. The method of claim 9, further comprising inhibiting the delivery of the second atrial C/D shock upon termination of the ERAF sustained duration.

11. The method of claim 7, further comprising:
detecting AF termination during time out of the ERAF sustained duration; and
terminating the ERAF sustained duration in response to detected AF termination.

12. The method of claim 11, further comprising inhibiting the delivery of the second atrial C/D shock upon termination of the ERAF sustained duration.

13. A method of decreasing incidences of early recurrence of atrial fibrillation (ERAF) following delivery of an atrial cardioversion/defibrillation (C/D) shock to a patient's heart by an implantable cardioverter/defibrillator responsive to a an atrial fibrillation (AF) episode comprising:
detecting one of an AF episode or a patient command generated by a patient activator operated by a patient experiencing symptoms of an atrial fibrillation (AF) episode;
delivering the atrial C/D shock to the patient's heart in response to the detected one of the AF episode or the patient command;
detecting ERAF, if present, following delivery of the atrial C/D shock;
timing out an ERAF sustained duration upon detecting ERAF during the ERAF detect time; and
inhibiting delivery of a further atrial C/D shock for the duration of the ERAF sustained duration.

14. A system for decreasing incidences of early recurrence of atrial fibrillation (ERAF) following delivery of an atrial cardioversion/defibrillation (C/D) shock to the atria of a patient's heart by an implantable cardioverter/defibrillator comprising:
means for detecting ERAF, if present, following delivery of an atrial C/D shock;
means for timing out an ERAF sustained duration upon detecting ERAF during the ERAF detect time; and
means for inhibiting delivery of a further atrial C/D shock for the duration of the ERAF sustained duration.

15. The system of claim 14, further comprising:
means for determining if atrial fibrillation (AF) is present upon time out of the ERAF sustained duration; and
means for delivering a further atrial C/D shock to the atria upon determining that AF is present upon time out of the ERAF sustained duration.

16. The system of claim 15, further comprising:
means for detecting AF termination during time out of the ERAF sustained duration; and
means for terminating the ERAF sustained duration in response to detected AF termination.

17. The system of claim 14, further comprising:
means for detecting AF termination during time out of the ERAF sustained duration; and
means for terminating the ERAF sustained duration in response to detected AF termination.

18. A system for decreasing incidences of early recurrence of atrial fibrillation (ERAF) following delivery of an atrial cardioversion/defibrillation (C/D) shock to a patient's heart by an implantable cardioverter/defibrillator responsive to a patient command generated by a patient activator operated by a patient experiencing symptoms of an atrial fibrillation (AF) episode comprising:
means for delivering the atrial C/D shock to the patient's heart in response to the patient command;
means for detecting ERAF, if present, following delivery of an atrial C/D shock;
means for timing out an ERAF sustained duration upon detecting ERAF during the ERAF detect time; and
means for inhibiting delivery of a further atrial C/D shock in response to a further patient command for the duration of the ERAF sustained duration.

19. The system of claim 18, further comprising means for alerting the patient that ERAF is detected and that delivery of the further atrial C/D commanded by the patient is inhibited.

20. A system for decreasing incidences of early recurrence of atrial fibrillation (ERAF) following delivery of an atrial cardioversion/defibrillation (C/D) shock to a patient's heart by an implantable cardioverter/defibrillator responsive to a an atrial fibrillation (AF) episode comprising:
means for detecting an AF episode;
means for delivering the atrial C/D shock to the atria of the patient's heart in response to the detected AF episode;
means for detecting ERAF, if present, following delivery of the atrial C/D shock;
means for timing out an ERAF sustained duration upon detecting ERAF during the ERAF detect time; and
means for delivering a further atrial C/D shock upon time out of the ERAF sustained duration.

21. The system of claim 20, further comprising:
means for determining if atrial fibrillation (AF) is present upon time out of the ERAF sustained duration; and
means for delivering the further atrial C/D shock to the atria only upon determining that AF is present upon time out of the ERAF sustained duration.

22. The system of claim 21, further comprising:
means for detecting AF termination during time out of the ERAF sustained duration; and
means for terminating the ERAF sustained duration in response to detected AF termination.

23. The system of claim 22, further comprising means for inhibiting the delivery of the second atrial C/D shock upon termination of the ERAF sustained duration.

24. The system of claim 20, further comprising:
means for detecting AF termination during time out of the ERAF sustained duration; and
means for terminating the ERAF sustained duration in response to detected AF termination.

25. The system of claim 24, further comprising means for inhibiting the delivery of the second atrial C/D shock upon termination of the ERAF sustained duration.

26. A system of decreasing incidences of early recurrence of atrial fibrillation (ERAF) following delivery of an atrial cardioversion/defibrillation (C/D) shock to a patient's heart by an implantable cardioverter/defibrillator responsive to a an atrial fibrillation (AF) episode comprising:
means for detecting one of an AF episode or a patient command generated by a patient activator operated by a patient experiencing symptoms of an atrial fibrillation (AF) episode;
means for delivering the atrial C/D shock to the patient's heart in response to the detected one of the AF episode or the patient command;
means for detecting ERAF, if present, following delivery of the atrial C/D shock;
means for timing out an ERAF sustained duration upon detecting ERAF during the ERAF detect time; and
means for inhibiting delivery of a further atrial C/D shock for the duration of the ERAF sustained duration.
